(19) United States
(12) Patent Application Publication
(10) Pub. No.: US 2020/0085865 A1
(43) Pub. Date: Mar. 19, 2020

(54) Intramammary Veterinary Composition

(71) Applicant: ZOETIS BROOMHILL IP LIMITED, Loughlinstown (IE)

(72) Inventors: Francisco Javier MOLINS ALBANELL, Dublin (IE); Brendan Gerard SMITH, Dublin (IE); Sandeep GUPTA, Wicklow (IE)

(21) Appl. No.: 16/690,379

(22) Filed: Nov. 21, 2019

Related U.S. Application Data
(63) Continuation of application No. PCT/EP2018/065230, filed on Jun. 8, 2018.

(30) Foreign Application Priority Data
Jun. 9, 2017 (EP) 17175215.7

(51) Int. Cl. A61K 33/30; A61K 47/69; A61K 9/00

(52) U.S. Cl. CPC A61K 33/30; A61K 45/06; A61K 9/0041; A61K 47/6903

(57) ABSTRACT

A seal formulation for forming a physical barrier in the teat canal of a non-human animal comprises zinc oxide in a gel base. The seal formulation contains at least 40% by weight of the zinc oxide.

### CROSS REFERENCE TO RELATED APPLICATIONS

This application is a continuation of International Application No. PCT/EP2018/065230, filed Jun. 8, 2018, which claims the benefit of European Patent Application No. 17175215.7, filed Jun. 9, 2017, both of which are expressly incorporated herein by reference in their entirety.

### INTRODUCTION

This invention relates to a seal formulation for forming a physical barrier in the teat canal.

An intra-mammary teat sealant containing bismuth subnitrate in a gel base is known. The teat sealant may be used in conjunction with an antibiotic for prophylaxis or treatment of mastitis as described in GB 2273441A. It is also known to use the teat sealant on its own as a prophylactic treatment to protect against ingress of pathogens during an animal's dry period (WO9826759A).

These known teat seals have been proven to be highly effective over many years. One potential issue however is that if, on completion of the protective period, all of the seal is not fully stripped out of the teat, small amounts of residual teat sealant containing bismuth subnitrate can present during subsequent milkings and can adhere to the milking machine lines.

It is a challenge to provide a teat sealant which can be readily formulated, delivered into the teat canal, which will form an effective seal whilst being reliably stripped out of the teat canal when no longer required.

### STATEMENTS OF INVENTION

According to the invention there is provided a seal formulation for forming a physical barrier in the teat canal of a non-human animal comprising zinc oxide in a gel base wherein the seal formulation contains at least 40% by weight of zinc oxide.

In one embodiment the seal formulation contains from 40% to 70% by weight of the zinc oxide.

In one embodiment the seal formulation contains from 45% to 65% by weight of the zinc oxide.

The seal formulation may contain approximately 55% by weight of the zinc oxide.

In one embodiment the seal formulation further comprises a thixotrophic agent.

In some cases the seal formulation contains from 0.1% to 1.5% of the thixotrophic agent.

The seal formulation may contain from 0.6 to 1.0% of the thixotrophic agent.

In one case the seal formulation contains approximately 0.8% of the thixotrophic agent.

In one embodiment the thixotrophic agent comprises colloidal anhydrous silica.

In one embodiment the base is a gel based on aluminium stearate.

The base may include liquid paraffin as a vehicle.

In one embodiment the seal formulation contains from 35% to 65% of the base.

In one embodiment the seal formulation contains from 40% to 45% % of the base.

The invention also provides a use or method of use of a seal formulation, comprising zinc oxide in a gel base, in the preparation of a medicament for forming a physical barrier in a teat canal for prophylactically controlling infection of the mammary gland in a non-human animal by a mastitis-causing organism.

In one embodiment said prophylaxis does not involve the use of an antibiotic.

In one case the seal formulation does not contain any other anti-infective.

30 In some cases the seal formulation contains from 40% to 70% by weight of the zinc oxide such as from 45% to 65% by weight of the zinc oxide, or such as approximately 55% by weight of the zinc oxide.

In one embodiment the seal formulation further comprises a thixotrophic agent.

The seal formulation may contain from 0.1% to 1.5% of the thixotrophic agent such as from 0.6 to 1.0% of the thixotrophic agent, such as approximately 0.8% of the thixotrophic agent.

In one case the thixotrophic agent comprises colloidal anhydrous silica.

In some cases the base is a gel based on aluminium stearate.

The base may include liquid paraffin as a vehicle.

The seal formulation may contain from 35% to 65% of the base such as from 40% to 45% of the base.

Zinc oxide has anti-infective properties and has been used effectively in the treatment of many skin disorders. Zinc oxide has a mild astringent and antiseptic action. Zinc oxide is a Category I skin protector, and promotes healthy skin. Zinc oxide is used for treatment of skin diseases and infections such as eczema, impetigo, ringworm, varicose ulcers, pruritus and psoriasis. It is believed that Zinc oxide regulates the activity of oil glands and is required for protein, DNA and RNA synthesis and collagen and other irritants.

The formulation includes a thixotrophic agent or rheology modifier or emulsifier. One such is fumed silica which is also known as anhydrous colloidal silica. It is available from Evonik under the Trade Name Aerosil. It is also available from Cabot Corporation (Cab-o-sil) and Wacker Chemie-Owens Corning and OCI (Konasil).

The invention provides a teat seal which contains an anti-infective and provides an effective physical barrier to the teat canal of cattle for the prevention of intramammary infections throughout the dry period.

The teat seal of the invention has the following properties
Non-toxic, biocompatible, and capable of being sterilised.
Persistent—the seal remains in situ for the duration of the dry cow period
Consistency—the seal does not break up within the teat
Ease of removal—at the end of the dry period the seal is easily removable from the udder and does not give rise to persistent residues of the seal
Radiopaque
Ease of delivery

### BRIEF DESCRIPTION OF THE FIGS.

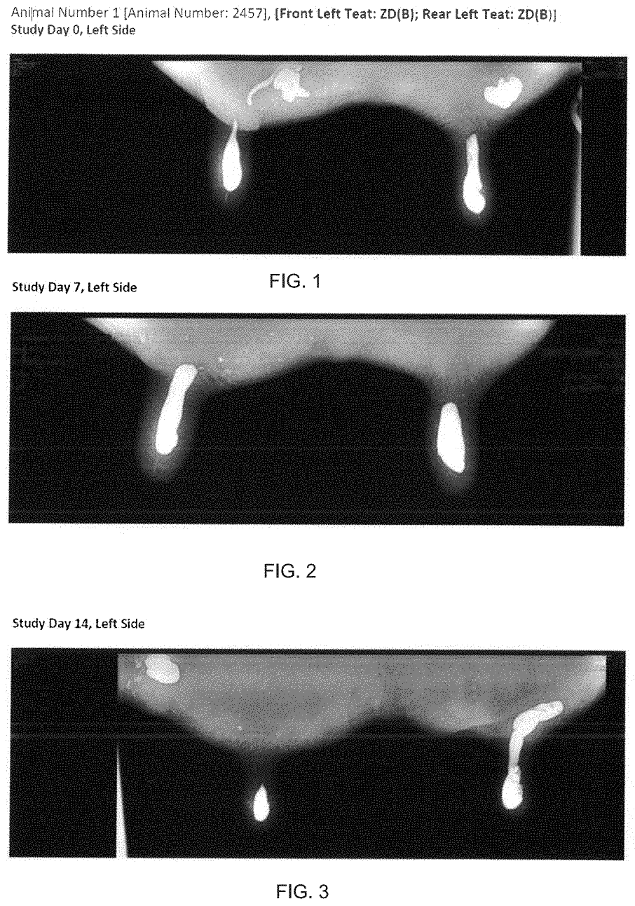

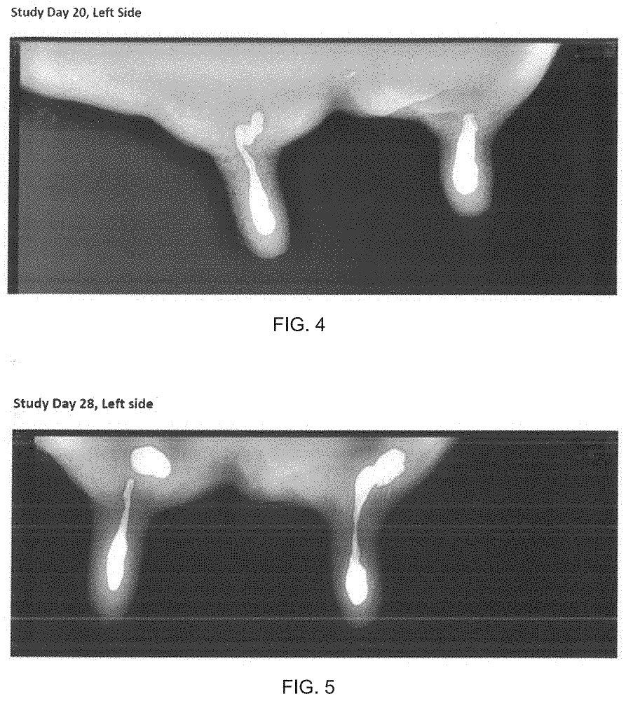

FIGS. 1 to 5 are X-ray images of the teats of a cow over a period after administration of a teat seal of the invention.

### DETAILED DESCRIPTION

The invention will be more clearly understood from the following description thereof given by way of example only.

### EXAMPLE 1

| | | |
|---|---|---|
| Component | Quantity per g | Quantity (% w/w) |

| | | | |
|---|---|---|---|
| Zinc Oxide | 547.0 | mg | 54.7% |
| Colloidal Anhydrous silica | 8.0 | mg | 0.8% |
| Aluminium di/tri stearate | 62.5 | mg | 6.25% |
| Liquid paraffin, Heavy | q.s. 1 | g | q.s. 100% |

The formulation above was prepared by the following process:

Liquid paraffin, heavy is added to a vessel.

Aluminium di-/tri stearate is added to the liquid paraffin, heavy, stirred and heated to a minimum of 130° C.

The mixture is maintained at this temperature for a minimum of 30 minutes.

The mixture is cooled to below 55° C.

While stirring, Zinc Oxide is added to the vessel and mixed until homogenous.

Colloidal anhydrous silica is then added and mixed until homogenous.

The product is then filled into intramammary syringes.

The filled syringes are sterilised by gamma irradiation at a minimum dose of 25 kGy.

### EXAMPLE 2

Two quarters of an uninfected cow were infused at drying off using a syringe containing the seal formulation of example 1.

X rays of the quarters were taken at days 0, 7, 14, 20 and 28 as shown in FIG. 1.

It will be seen form these radiographs that the seal persisted very well in the teats throughout the period of investigation thus ensuring that the teats remained sealed off against any potential bacterial ingress.

After day 28 the seals were stripped from the quarters and were harvested into sample containers for examination. The recovered seal, when compared to its appearance and characteristics pre-infusion, was found to have maintained its appearance and consistency.

The invention is not limited to the embodiments hereinbefore described, which may be varied in detail.

1. A seal formulation for forming a physical barrier in the teat canal of a non-human animal comprising zinc oxide in a gel base wherein the seal formulation contains at least 40% by weight of zinc oxide.

2. A seal formulation as claimed in claim 1 wherein the seal formulation contains from 40% to 70% by weight of the zinc oxide.

3. A seal formulation as claimed in claim 1 which further comprises a thixotrophic agent.

4. A seal formulation as claimed in claim 3 wherein the seal formulation contains from 0.1% to 1.5% of the thixotrophic agent.

5. A seal formulation as claimed in claim 3 wherein the seal formulation contains from 0.6 to 1.0% of the thixotrophic agent.

6. A seal formulation as claimed in claim 3 wherein the seal formulation contains approximately 0.8% of the thixotrophic agent.

7. A seal formulation as claimed in claim 3 wherein the thixotrophic agent comprises colloidal anhydrous silica.

8. Use of a seal formulation, comprising zinc oxide in a gel base, in the preparation of a medicament for forming a physical barrier in a teat canal for prophylactically controlling infection of the mammary gland in a non-human animal by a mastitis-causing organism.

9. Use as claimed in claim 8 wherein said prophylaxis does not involve the use of an antibiotic.

10. Use as claimed in claim 8 wherein the seal formulation does not contain any other anti-infective.

11. Use as claimed in claim 8 wherein the seal formulation contains from 40% to 70% by weight of the zinc oxide.

12. Use as claimed in claim 8 wherein the seal formulation further comprises a thixotrophic agent.

13. Use as claimed in claim 12 wherein the seal formulation contains from 0.1% to 1.5% of the thixotrophic agent.

14. Use as claimed in claim 12 wherein the seal formulation contains from 0.6 to 1.0% of the thixotrophic agent.

15. Use as claimed in claim 12 wherein the seal formulation contains approximately 0.8% of the thixotrophic agent.

16. Use as claimed in claim 12 wherein the thixotrophic agent comprises colloidal anhydrous silica.

17. Use as claimed in claim 8 wherein the base is a gel based on aluminium stearate.

18. Use as claimed in claim 8 wherein the base includes liquid paraffin as a vehicle.
